(19) United States
(12) Patent Application Publication
(10) Pub. No.: US 2024/0385319 A1
(43) Pub. Date: Nov. 21, 2024

(54) ULTRASOUND IMAGING METHOD, COMPUTER DEVICE, AND ULTRASOUND IMAGING SYSTEM

(71) Applicant: United Imaging Research Institute of Intelligent Imaging, Beijing (CN)

(72) Inventors: Jian An, Beijing (CN); Fanglue Lin, Beijing (CN)

(21) Appl. No.: 18/668,285

(22) Filed: May 20, 2024

(30) Foreign Application Priority Data
May 18, 2023 (CN) 202310567655.6

(51) Int. Cl. G01S 15/89; A61B 8/00; A61B 8/06; A61B 8/08; G01S 7/52

(52) U.S. Cl. CPC G01S 15/8959; A61B 8/06; A61B 8/4483; A61B 8/5207; G01S 7/52077; G01S 7/52085; G01S 7/52046

(57) ABSTRACT

An ultrasound imaging method, a computer device and an ultrasound imaging system. The ultrasound imaging method includes: encoding initial ultrasound pulsed waves to obtain target ultrasound pulsed waves, and obtaining multiple sets of ultrasound echo data of a to-be-imaged area based on the target ultrasound pulsed waves, the number of cycles of each of the target ultrasound pulsed waves being greater than the number of cycles of each of the initial ultrasound pulsed waves; and decoding the multiple sets of the ultrasound echo data to obtain an ultrasound image sequence. The ultrasound imaging method can improve an effect of micro-blood flow imaging for deep tissues.

### CROSS-REFERENCE TO RELATED APPLICATIONS

The present application claims priority of the Chinese patent application No. 202310567655.6, filed on May 18, 2023, entitled “Ultrasound Imaging Method and Apparatus, Computer Device, and Ultrasound Imaging System”, the entire content of which is incorporated herein by reference.

### TECHNICAL FIELD

The present disclosure relates to the field of imaging technology, and in particular to an ultrasound imaging method and apparatus, a computer device and an ultrasound imaging system.

### BACKGROUND

In related ultrasound blood flow imaging technologies, the structure and distribution of low-velocity minute vessels cannot be observed due to limitations such as high noise and low frame rate. In recent years, unfocused ultrasound imaging technology based on plane waves or divergent waves has greatly increased the data sampling frequency, and a higher frequency means obtaining richer information. Together with advanced tissue clutter filters, the ultrasonic micro-blood flow imaging for superficial tissues has been widely used gradually.

However, due to a weak penetrating ability of an unfocused wave in a deeper portion of the tissue, the echo signals of a blood flow will inevitably be greatly weakened or even submerged in electronic noises. Ultrasound imaging methods in related technologies have problems such as poor effect of micro-blood flow imaging for deep tissues.

### SUMMARY

The present disclosure provides an ultrasound imaging method, a computer device and an ultrasound imaging system to improve an effect of micro-blood flow imaging for deep tissues.

In a first aspect, the present disclosure provides an ultrasound imaging method. The method includes: encoding initial ultrasound pulsed waves to obtain target ultrasound pulsed waves, and obtaining multiple sets of ultrasound echo data of a to-be-imaged area based on the target ultrasound pulsed waves, the number of cycles of each of the target ultrasound pulsed waves being greater than the number of cycles of each of the initial ultrasound pulsed waves; and decoding the multiple sets of the ultrasound echo data to obtain an ultrasound image sequence.

In an embodiment, the to-be-imaged area includes a target area. The method further includes: performing cyclic polling-based coherent compounding on the ultrasound image sequence to obtain a plurality of target image sequences, each of the plurality of target image sequences including multiple single-angle unfocused wave images that are sequentially adjacent in the ultrasound image sequence; and processing the plurality of target image sequences separately for the target area to obtain a target-area image sequence capable of imaging the target area.

In an embodiment, performing the cyclic polling-based coherent compounding on the ultrasound image sequence to obtain the plurality of target image sequences includes: determining a current target image sequence by selecting from single-angle unfocused wave images in the ultrasound image sequence; and determining a next target image sequence by selecting from the single-angle unfocused wave images in the ultrasound image sequence based on the current target image sequence, until the plurality of target image sequences are obtained. The next target image sequence includes at least one of single-angle unfocused wave images in the current target image sequence.

In an embodiment, the to-be-imaged area further includes a related area. The method further includes: performing coherent compounding on multiple sets of complex image sequences in the ultrasound image sequence to obtain relation image sequences, each set of complex image sequences including multiple single-angle unfocused wave images; and processing the relation image sequences separately for the related area to obtain a related-area image sequence capable of imaging the related area.

In an embodiment, the method further includes: splicing the target-area image sequence and the related-area image sequence to obtain a to-be-imaged-area complex image sequence.

In an embodiment, the method further includes: performing imaging on the to-be-imaged-area complex image sequence to obtain a to-be-imaged-area ultrasound image.

In an embodiment, performing imaging on the to-be-imaged-area complex image sequence to obtain the to-be-imaged-area ultrasound image includes: multiplying the to-be-imaged-area complex image sequence by a conjugate thereof; and summing a product of the to-be-imaged-area complex image sequence and the conjugate thereof along the time dimension, and performing a logarithmic compression to obtain the to-be-imaged-area ultrasound image.

In an embodiment, processing the plurality of target image sequences separately for the target area includes: performing random singular value decomposition filtering on the plurality of target image sequences separately for the target area. Processing the relation image sequences separately for the related area, includes: performing singular value decomposition filtering on the relation image sequences separately for the related area.

In an embodiment, encoding the initial ultrasound pulsed waves to obtain the target ultrasound pulsed waves, including: obtaining a Walsh matrix; forming a waveform encoding matrix based on the Walsh matrix and delays of channels of the initial ultrasound pulsed waves; and encoding the initial ultrasound pulsed waves based on the waveform encoding matrix to obtain the target ultrasound pulsed waves.

In an embodiment, before forming the waveform encoding matrix based on the Walsh matrix and the delays of channels of the initial ultrasound pulsed waves, the method further includes: determining the delays of channels of the initial ultrasound pulsed waves based on the number of transmission angles and a maximum transmission angle of the initial ultrasound pulsed waves.

In an embodiment, the Walsh matrix is as follows:

$$W ( p , q ) = {\prod }_{k = 0}^{M - 1} {[ R ( k + 1 , q ) ]}^{{g ( p )}_{k}}$$

where R(k+1,q) denotes a Rademaker function, g(p) denotes a Gray code of p, g(p)ₖ denotes a k-th bit of the Gray code g(p), p denotes an index number of the number of times the initial ultrasound pulsed waves transmit, p=1, 2 to 2^{na}, na denotes any one of 1, 2, 3or 4, M denotes an order of the Walsh matrix, M=2^{na}, and q denotes a continuous time variable.

In an embodiment, the target ultrasound pulsed waves are as follows:

$$TWB ( i , p ) [ ( 1 + ( j - 1 ) \times {N}_{TW} ) to j \times {N}_{TW} ] = TWA ( i , j ) \times W ( p , q ) ,$$

where TWB(i, p) denotes the target ultrasound pulsed waves, i denotes an index number of an array element of a transducer, j denotes an index number of an initial transmission angle, TWA(i, j) denotes the initial ultrasound pulsed waves, and N_{TW} denotes a length of the initial ultrasound pulsed waves TWA(i, j).

In an embodiment, the order M of the Walsh matrix is equal to the number of transmission angles of the initial ultrasound pulsed waves.

In an embodiment, encoding the initial ultrasound pulsed waves based on the waveform encoding matrix to obtain the target ultrasound pulsed waves includes: encoding each channel of the initial ultrasound pulsed waves based on the waveform encoding matrix to obtain a transmission waveform of each channel; and obtaining the target ultrasound pulsed waves based on the transmission waveform of each channel.

In an embodiment, decoding the multiple sets of the ultrasound echo data to obtain the ultrasound image sequence includes: obtaining an inverse matrix of the waveform encoding matrix to serve as a waveform decoding matrix; decoding each set of the multiple sets of ultrasound echo data based on the waveform decoding matrix to obtain multiple sets of radio frequency (RF) data, respectively; and performing beamforming on each set of the multiple sets of RF data based on transmission parameters of the initial ultrasound pulsed waves to obtain the ultrasound image sequence.

In an embodiment, the waveform decoding matrix is the inverse matrix of the Walsh matrix.

The multiple sets of RF data are as follows:

$$RFB ( i , p ) = {\sum }_{j = 1}^{M} RFA ( i , j ) \times {W}^{′} ( p , j )$$

where RFB(i, p) denotes the RF data, RFA(i, j) denotes the ultrasound echo data, and W′(p,j) denotes the inverse matrix of the Walsh matrix.

In an embodiment, the number of the transmission angles of the initial ultrasound pulsed waves is 2^{na}. na is any one of 1, 2, 3 or 4; and the maximum transmission angle is any value from 3 degrees to 24 degrees.

In a second aspect, the present disclosure provides a non-transitory computer-readable storage medium, including a computer program stored thereon. The computer program, when executed by a processor, causes the processor to perform steps of the method above.

In a third aspect, the present disclosure provides a computer device, including a memory and a processor. The memory stores a computer program, and the processor, when executing the computer program, performs steps of the method above.

In a fourth aspect, the present disclosure provides an ultrasound imaging system, including: a waveform generator and an ultrasonic transducer, which are configured for generating and transmitting target ultrasound pulsed waves; and a computer device connected to the waveform generator and the ultrasonic transducer. The computer device, when executing a computer program, performs steps of the method.

According to the ultrasound imaging method and apparatus, the computer device, and the ultrasound imaging system above, by encoding the initial ultrasound pulsed waves, the target ultrasound pulsed waves are obtained. The multiple sets of ultrasound echo data of the to-be-imaged area are obtained based on the target ultrasound pulsed waves, and the target ultrasound pulsed waves are obtained. Compared with the initial ultrasound pulsed waves, the obtained target ultrasound pulsed waves each have an increased number of cycles. The target ultrasound pulsed waves configured for transmission can increase the number of cycles of the transmission pulse, thus greatly improving the penetration ability of unfocused waves in the deeper parts of the tissue and preventing the micro-blood flow signals in the deeper parts from being submerged in noises. The obtained signals of ultrasound echo data corresponding to the target ultrasound pulsed waves are enhanced, so that the intensity of the ultrasound echo data which reflect the echo signals of the blood flow is increased at the front end, the noise level is inhibited, and the signal-enhanced ultrasound echo data is obtained. By decoding the multiple sets of the ultrasound echo data to obtain the ultrasound image sequence, the intensity of the echo signals of each individual angle can be enhanced after echo decoding, and it is ensured that the axial resolution of the image is not affected by the increase in the number of cycles of the transmission pulse. The ultrasound image sequence may be used for the subsequent coherent compounding and imaging, thereby improving the effect of the micro-blood flow imaging for the deep tissues

### BRIEF DESCRIPTION OF THE DRAWINGS

FIG. 1 is a schematic flow chart of an ultrasound imaging method according to an embodiment of the present disclosure;

FIG. 2 is a schematic flow chart of an ultrasound imaging method according to another embodiment of the present disclosure;

FIG. 3 is a schematic flow chart of an ultrasound imaging method according to yet another embodiment of the present disclosure;

FIG. 4 is a schematic flow chart of an ultrasound imaging method according to yet another embodiment of the present disclosure;

FIG. 5(a) shows conventional coherent compounding according to an embodiment of the present disclosure;

FIG. 5(b) shows cyclic polling-based coherent compounding according to an embodiment of the present disclosure;

FIG. 6 is a schematic flow chart of ultrasound imaging steps according to an embodiment of the present disclosure;

FIG. 7 is a schematic flow chart of ultrasound imaging steps according to another embodiment of the present disclosure;

FIG. 8 is a schematic flow chart of ultrasound imaging steps according to yet another embodiment of the present disclosure;

FIG. 9 is a schematic flow chart of ultrasound imaging steps according to another embodiment of the present disclosure;

FIG. 10(a) shows initial ultrasound pulsed waves according to an embodiment of the present disclosure;

FIG. 10(b) shows target ultrasound pulsed waves according to an embodiment of the present disclosure;

FIG. 10(c) shows ultrasound echo data according to an embodiment of the present disclosure;

FIG. 10(d) shows radio frequency (RF) data according to an embodiment of the present disclosure;

FIG. 11(a) is a schematic flow chart of an ultrasound imaging method according to an example of the present disclosure;

FIG. 11(b) is a schematic flow chart of the ultrasound imaging method according to another example of the present disclosure;

FIG. 12 shows a B-scan ultrasonography image of an adult kidney according to an embodiment of the present disclosure;

FIG. 13(a) shows an ultrasound blood flow image of the kidney obtained by a conventional method in the prior art;

FIG. 13(b) shows an ultrasonic micro-blood flow image of the kidney according to an embodiment of the present disclosure;

FIG. 14 is a block diagram showing a structure of an ultrasound imaging device according to an embodiment of the present disclosure; and

FIG. 15 shows an internal structure of a computer device according to an embodiment of the present disclosure.

### DETAILED DESCRIPTION OF THE EMBODIMENTS

In order to make the purpose, technical solutions and advantages of the present disclosure clearer and better understood, the present disclosure will be further described in detail below with reference to the accompanying drawings and embodiments. It should be understood that the specific embodiments described herein are only used to explain the present disclosure but not intended to limit the present disclosure.

It should be noted that, in the early wall filtering technology that continued conventional ultrasound Doppler imaging, low-pass or band-pass filters as clutter inhibition methods are used to filter several consecutive frames of ultrasound RF data along the slow-time signal direction. However, its detection of micro-blood flow is often not ideal due to tissue movement and noise. Related technologies of ultrasonic blood flow imaging have limitations such as high noise and low frame rate, and cannot observe the structure and distribution of low flow rates and micro-blood vessels. The detection and imaging of micro-blood vessels play a vital role in assessing the physiological status of the human body.

The development of unfocused ultrasound imaging technology of plane waves or divergent waves has greatly increased the data sampling frequency. Plane wave ultrasound imaging uses plane wave transmission, and a single transmission can cover the entire to-be-imaged area. Compared with line scan focused ultrasound in the related art, the plane wave ultrasound imaging can greatly reduce the number of times the one frame of image is transmitted, thus significantly improving the scanning frame rate. However, the resolution and signal to noise ratio (SNR) of the plane wave image obtained by a single transmission are severely reduced. Using ultra-high frame rates of unfocused waves can greatly highlight the difference between tissue and blood flow, and can increase the detectable range of the minimum blood flow velocity generated by the Doppler effect, thus detecting tiny blood flow signals and vascular structures. According to a multi-angle coherent plane-wave compounding (CPWC) method, phases of the pulses transmitted by different array elements of the ultrasonic transducer are delayed to generate plane waves having different angles relative to the transducer, the echo signals reflected by the plane waves of various angles are collected, and multiple images are superimposed by using coherent compounding. According to the multi-angle CPWC method, the number of compound angles is increased, and noise is inhibited, thereby effectively improving the resolution and the SNR of the image, improving the quality of the plane waves imaging. Compared with conventional focused waves, in the case of equivalent image quality, the method greatly improves the frame rate. Ultrasonic micro-blood flow imaging based on CPWC and feature decomposition can be widely used in blood flow detection for small animals and superficial parts of the human body.

However, a limitation of the CPWC is that as the total number of plane wave transmission angles increase, the imaging frame rate decreases accordingly. When the imaging depth is relatively small, the time required to receive echo data of a plane wave each time is shorter, and the frame rate is not seriously affected by the total number of plane wave transmission angles. For deep tissue imaging, the time required to receive echo data of a plane wave each time has increased significantly. In this case, if the number of transmission angles is increased, the final imaging frame rate will be greatly reduced, which will result in a severe reduction in a sensitivity of detecting the micro-blood flow, and it is difficult to balance the signal noise level and the sensitivity of detecting the micro-blood flow. Another strategy that may effectively improve the intensity of echo signals of the deep blood flow is to increase the number of cycles of the transmission pulse. For example, in an ultrasonic Doppler blood flow imaging in the related art, lower-frequency transmission ultrasonic pulses having a much higher number of cycles than those in a conventional imaging mode are usually used, so as to overcome the attenuation of the echo signals of the blood flow during a transmission in tissue. However, as the number of cycles of the transmission pulse increases, the axial resolution of the image will inevitably decrease, making it difficult to achieve high-resolution imaging for tiny vessels. In summary, due to the weak penetrating ability of unfocused waves in deeper parts of the tissue, especially in abdominal parenchymal organs located in the deeper parts of the body, such as in a liver and a kidney, the echo signals of the blood flow will inevitably be weakened greatly and even submerged in electronic noises. The challenges to be faced in detecting the micro-blood flow in the abdominal parenchymal organs are more prominent.

In recent years, methods based on a feature composition such as a singular value decomposition (SVD) or a principal component analysis (PCA) have been widely used in ultrafast ultrasonic microvascular imaging. Conventional clutter filters only operate in the time dimension, while a method, such as the SVD, takes advantage of different characteristics of a tissue and a blood movement in terms of spatiotemporal coherence, providing higher-dimensional feature information and greatly improving the sensitivity level for the tiny blood flow. However, because the method only relies on weak signals from the movement of red blood cells, this method has poor noise immunity. When interferences of noises or artifacts are large, the sensitivity for the micro vessels imaging will be greatly reduced.

According to conventional experience, increasing the number of cycles of transmission ultrasonic pulses is an effective way to increase the penetration depth of the unfocused waves, but at the same time it will result in a substantial decrease in the axial resolution of the image. Increasing the number of transmission angles of the unfocused waves and using coherent compounding is also a conventional noise inhibiting method, but this method is at the expense of reducing the final imaging frame rate, which is especially prominent in deep imaging and further reduces the sensitivity for detecting the micro-blood flow. In addition, the abdominal parenchymal organs move slowly due to heartbeat and respiration, and too many transmission angles will introduce new clutter artifacts during the coherent compounding. In summary, conventional methods for enhancing echo signals of the micro-blood flow and inhibiting noises are difficult to work in imaging deep parts such as abdominal parenchymal organs.

Therefore, the ultrasonic blood flow imaging methods in the related art are not applicable to the detection and the micro-blood flow imaging for deep tissues and organs of the human body. An ultrasound imaging method applicable to a detection for the micro-blood flow in abdominal parenchymal organs needs to be proposed urgently.

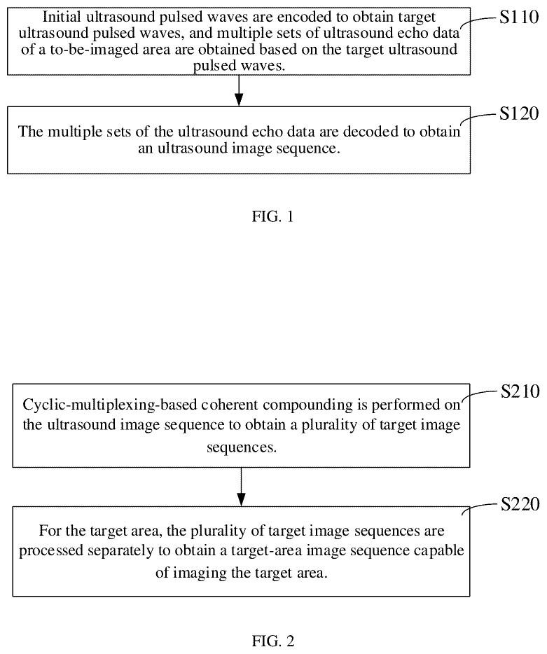

In an embodiment, as shown in FIG. 1, the present disclosure provides an ultrasound imaging method. The ultrasound imaging method of the present application may be applied in an ultrasound imaging system, and includes following steps.

In step S110, initial ultrasound pulsed waves are encoded to obtain target ultrasound pulsed waves, and multiple sets of ultrasound echo data of a to-be-imaged area are obtained based on the target ultrasound pulsed waves. The number of cycles of each of the target ultrasound pulsed waves is greater than the number of cycles of each of the initial ultrasound pulsed waves.

Specifically, the initial ultrasound pulsed waves can be encoded to determine the target ultrasound pulsed waves. The number of cycles of each of the target ultrasound pulsed waves obtained by encoding is greater than the number of cycles of each of the initial ultrasound pulsed waves. Each of the initial ultrasound pulsed wave may be a multi-angle coherent compounding unfocused wave, and each of the multi-angle unfocused wave may include at least one of a multi-angle coherent compounding plane wave and a multi-angle divergent wave. Furthermore, the target ultrasound pulsed waves may be used as the waveform transmitted by each channel, multiple sets of ultrasonic pulses are transmitted in sequence, and the received echo data corresponding to the target ultrasound pulsed waves are saved, to obtain multiple sets of ultrasound echo data. The multiple sets of ultrasound echo data may be the obtained raw RF data. Through the above method of encoding the initial ultrasound pulsed waves to obtain the target ultrasound pulsed waves, the number of cycles of each transmission pulsed wave can be increased, thereby greatly improving the penetration ability of the unfocused waves in deeper parts of the tissues, for example, the parts at a depth greater than 12 cm, and preventing micro-blood flow signals in the deeper parts from being submerged in noise. By obtaining the ultrasound echo data corresponding to the target ultrasound pulsed waves, the intensity of the ultrasound echo data which reflect the blood flow echo signals is increased at the front end, i.e., before the wall filtering, and the noise level is inhibited.

In some examples, the target ultrasound pulsed waves may be determined based on the initial ultrasound pulsed waves and a waveform coding matrix. The waveform coding matrix may be determined based on the principle of code division multiplexing (CDM), and the target ultrasound pulsed waves may be determined based on the output of the convolution of the initial ultrasound pulsed waves and the waveform coding matrix. The waveform encoding matrix may be an M-order matrix. For example, the order M may be 2^{na}. The order M of the waveform encoding matrix may be determined based on the number of transmission plane-wave angles. The initial ultrasound pulsed waves may be processed through the waveform encoding matrix, for example, convolution processing, and a total length of the obtained target ultrasound pulsed wave is several times, for example, M times, longer than the length of the initial ultrasound pulsed wave, where M is the order of the waveform encoding matrix times, i.e., the target ultrasound pulsed wave can be divided into M segments in time sequence, and each segment has the same length as the initial ultrasound pulsed wave. The number of cycles of each of the target ultrasound pulsed wave is greater than the number of cycles of each of the initial ultrasound pulsed wave, where the number of cycles of each of the target ultrasound pulsed waves is equal to the number of cycles of the initial ultrasound pulsed wave multiplied by the order of the waveform encoding matrix. Then the target ultrasound pulsed waves, obtained based on the waveform encoding matrix and the initially ultrasound pulsed waves, are configured for transmission. The target ultrasound pulsed waves, compared with the initial ultrasound pulsed waves, improve the number of cycles of the transmission pulses. Furthermore, the determined target ultrasound pulsed waves may be used as the waveform transmitted by each channel, and the multiple sets of the target ultrasound pulsed waves may be transmitted in sequence, and multiple sets of received echo data corresponding to the target ultrasound pulsed waves are saved, to obtain multiple sets of ultrasound echo data. The multiple sets of ultrasound echo data can be the obtained raw RF data.

In some examples, the target ultrasound pulsed waves may be determined based on preset transmission parameters and the waveform encoding matrix. The waveform encoding matrix may be a matrix that encodes waveform data. For example, the initial ultrasound pulsed waves may be generated based on transmission parameters of the multi-angle coherent compounding unfocused waves. The transmission parameters may include at least one of the number of transmission angles, a maximum transmission angle, a center frequency of the transmission pulse, the number of cycles of the transmission pulse, and an initial phase of the transmission pulse. The waveform encoding matrix may be obtained according to a convolution of delays of all channels corresponding to the transmission inclination angles of the unfocused waves and the Walsh matrix.

In step S120, the multiple sets of the ultrasound echo data are decoded to obtain an ultrasound image sequence.

Specifically, the multiple sets of the ultrasound echo data may be decoded separately to obtain an ultrasound image sequence. The ultrasound image sequence may include multiple single-angle unfocused wave images. The single-angle unfocused wave images may include at least one of single-angle plane wave images or single-angle divergence wave images. The single-angle unfocused wave image can be complex image. By decoding each set of the ultrasound echo data, signal enhanced ultrasound image sequence is obtained for subsequent coherent compounding, thereby improving the effect of micro-blood flow imaging for deep tissues.

In some examples, the multiple sets of ultrasound echo data may be decoded separately based on a waveform decoding matrix corresponding to the waveform encoding matrix, to obtain the ultrasound image sequence. The waveform decoding matrix may be a matrix used to decode waveform data, and the waveform decoding matrix may be used for decoding the multiple sets of obtained ultrasound echo data separately, which can enhance the intensity of the echo signal at each individual angle after echo decoding, and ensure that the axial resolution of the image is not affected by the increase in the number of cycles of the transmission pulse, thereby obtaining signal-enhanced ultrasound image sequence.

In some examples, the multiple sets of obtained ultrasound echo data are decoded separately based on the waveform decoding matrix, to obtain decoded RF data. The waveform decoding matrix may be a matrix obtained based on the waveform encoding matrix and used to decode the waveform data. Beamforming may be performed on each set of decoded ultrasound echo data based on the transmission parameters. For example, the beamforming may be performed on the decoded RF data based on the transmission parameters, to obtain the ultrasound image sequence. The ultrasound image sequence may include N_{F} sets of complex image sequences IQData(t) arranged in sequence, where t=1,2,3 . . . or N_{F}, and a range of N_{F} may be from 50 to 300. In an embodiment, N_{F}=100. Each set of complex image sequences IQData(1) may include 2^{na} single-angle unfocused wave images IQData(p,t), where p=1,2,3 . . . or 2^{na}, where 2^{na} is the number of transmission angles among the transmission parameters, i.e., the single-angle unfocused wave images IQData(p,t) in each set of complex image sequence IQData(t) may be sorted according to an index number p of the number of times of transmissions. In an embodiment, 2^{na}=4.

According to the method of the embodiment of the present disclosure, the multiple sets of ultrasound echo data of the to-be-imaged area are obtained. The target ultrasound pulsed waves corresponding to the ultrasound echo data are obtained by encoding the initial ultrasound pulsed waves. Compared with the initial ultrasound pulsed waves, the obtained target ultrasound pulsed waves each have an increased number of cycles. The target ultrasound pulsed waves configured for transmission can increase the number of cycles of the transmission pulse, thus greatly improving the penetration ability of unfocused waves in the deeper parts of the tissue and preventing the micro-blood flow signals in the deeper parts from being submerged in noises. The obtained signals of ultrasound echo data corresponding to the target ultrasound pulsed waves are enhanced, so that the intensity of the ultrasound echo data which reflect the echo signals of the blood flow is increased at the front end, the noise level is inhibited, and the signal-enhanced ultrasound echo data is obtained. By decoding the multiple sets of the ultrasound echo data to obtain the ultrasound image sequence, the intensity of the echo signals of each individual angle can be enhanced after echo decoding, and it is ensured that the axial resolution of the image is not affected by the increase in the number of cycles of the transmission pulse. The ultrasound image sequence may be used for the subsequent coherent compounding and imaging, thereby improving the effect of the micro-blood flow imaging for the deep tissues.

In an embodiment, as shown in FIG. 2, the to-be-imaged area includes a target area. The ultrasound imaging method further described below.

In step S210, cyclic polling-based coherent compounding is performed on the ultrasound image sequence to obtain a plurality of target image sequences. Each target image sequence includes multiple single-angle unfocused wave images that are sequentially adjacent in the ultrasound image sequence.

In step S220, for the target area, the plurality of target image sequences are processed separately to obtain a target-area image sequence capable of imaging the target area.

In some embodiments, the ultrasound image sequence may include multiple single-angle unfocused wave images arranged in sequence. The cyclic polling-based coherent compounding may be performed on each single-angle unfocused wave image in the ultrasound image sequence to obtain the plurality of target image sequences. Each target image sequence may include multiple single-angle unfocused wave images that are sequentially adjacent in the ultrasound image sequence. The cyclic polling-based coherent compounding is performed on the ultrasound image sequence until all sets of selected complex image sequences are processed, so that the target image sequence is obtained. Further, for the target area, the plurality of target image sequences are processed separately, for example, the plurality of target image sequences are filtered to obtain a target-area image sequence. The target-area image sequence may be configured for imaging the target area. Through the cyclic polling-based coherent compounding, the number of the target image sequences may be selected according to actual needs, and the number of the target image sequences may be increased by multiplexing. For example, each target image sequence may include one set of complex image sequence, and each set of complex image sequence may include multiple single-angle unfocused wave images arranged in sequence, and part of the single-angle unfocused wave images in a current set of complex image sequence are the same as part of the single-angle unfocused wave images in a previous set of complex image sequence, such that the number of the target image sequences is greater than the number of sets of complex image sequences in the ultrasound image sequence. For the target area, target image sequences are processed separately to obtain a target-area image sequence capable of imaging in the target area, such that the frame rate of the target-area image sequence may be improved. Finally, the target-area image sequence with enhanced signals, low noise, and a high frame rate, which reflects the echo data of blood flow, is obtained. Compared with conventional methods, the ultrasound imaging method of the present application improves the signal strength and reduces the noise level, and may configure the number of the target image sequences according to actual needs to increase the frame rate, thus improving the effect of the micro-blood flow imaging for deep tissues.

In some examples, the ultrasound image sequence may be processed by multiplexing, to obtain the target image sequences IQDataH(t), where t=1, 2, 3, . . . , (2^{na}×(N_{F}−1)+1). Each target image sequence IQDataH(t) includes multiple single-angle unfocused wave images IQData(p,t) that are sequentially adjacent in ultrasound image sequence. The number of the target image sequences may be greater than the number of sets of the complex image sequences in the ultrasound image sequence. For example, the number N_{F} of sets of complex image sequences in the ultrasound image sequence may be 100, and the number of single-angle unfocused wave images in each set of the complex image sequence is 2^{na}=4. The number of the target image sequences IQDataH(t) selected from the ultrasound image sequence is 397,which is greater than 100. At least two sets of the target image sequences include the same single-angle unfocused wave image, thereby improving the frame rate of the target-area image sequence obtained after performing coherent compounding on the target image sequences, where the frame rate is finally used to reflect the clarity of the target-area image.

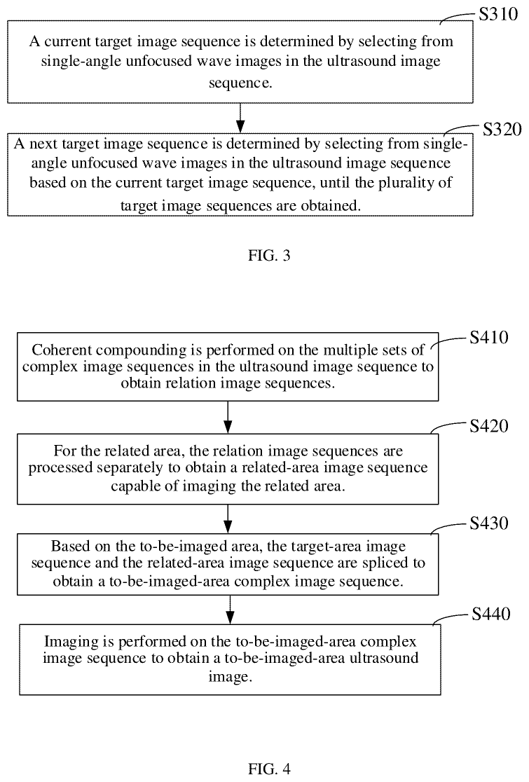

In an embodiment, as shown in FIG. 3, the step S210 of performing cyclic polling-based coherent compounding on the ultrasound image sequence to obtain the plurality of target image sequences, includes step S310 and step S320.

In step S310, a current target image sequence is determined by selecting from single-angle unfocused wave images in the ultrasound image sequence.

In step S320, a next target image sequence is determined by selecting from single-angle unfocused wave images in the ultrasound image sequence based on the current target image sequence, until the plurality of target image sequences are obtained. The next target image sequence includes at least one of the single-angle unfocused wave images in the current target image sequence.

Specifically, the ultrasound image sequence may include N_{F} sets of the complex image sequences IQData(t) arranged in sequence, where 1=1,2,3 . . . , N_{F}. Each set of the complex image sequence IQData(t) may include 2^{na} a single-angle unfocused wave images IQData(p,t). Each target image sequence can be determined by selecting sequentially, from the single-angle unfocused wave images IQData(p,t) in the ultrasound image sequence IQData(t) by setting a step size. During a selection of the target image sequences, the step size represents the number of jumped single-angle unfocused wave images between adjacent target image sequences, which may be less than the number of the single-angle unfocused wave images in the target image sequence and greater than 0, to obtain the plurality of target image sequences.

Further, compared with that obtained by the conventional methods, the target-area image sequence obtained based on the selected target image sequences have an improved frame rate. In this embodiment of the present disclosure, at least one of the single-angle unfocused wave images in the currently selected target image sequence is included in the next target image sequence, such that the cyclic polling-based coherent compounding is performed on the single-angle unfocused wave images, thereby increasing the frame rate of the target image sequences for imaging the target area.

In some examples, in the ultrasound image sequence IQData(t), where 1=1, 2, 3, . . . , N_{F}, that is, tis in a range from 1 to N_{F}. The number N_{F} of sets of the complex image sequences may be 100, and the number of the single-angle unfocused wave images included in each set is 2^{na}=4. In the ultrasound image sequence, one single-angle unfocused wave image may be configured as the step size for jumping to select the target image sequence, and four adjacent single-angle unfocused wave images may be selected sequentially to obtain 397 sets of the target image sequences, which is calculated by 2^{na}×(N_{F}−1)+1=397. That is, two adjacent target image sequences each include the same three single-angle unfocused wave images.

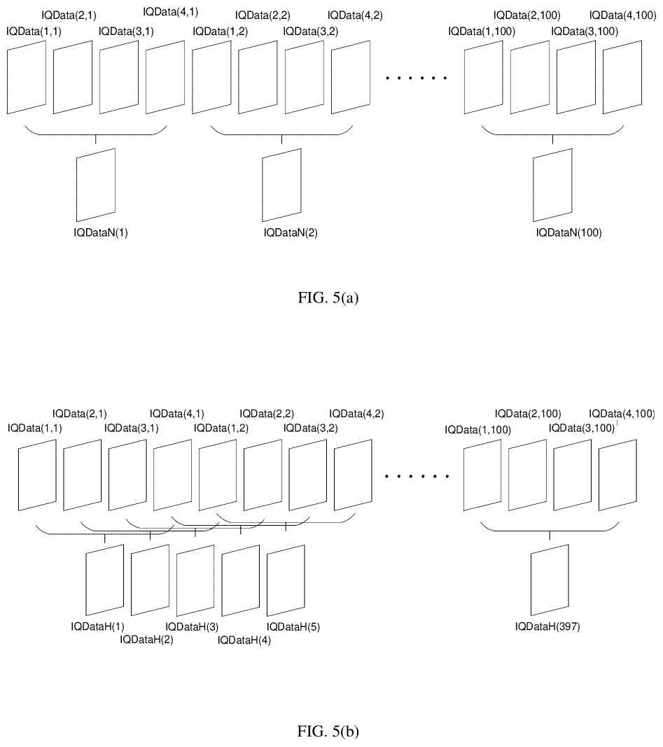

In an embodiment, the to-be-imaged area further includes a related area. The to-be-imaged-target volume in the target area is smaller than the to-be-imaged-target volume in the related area. The ultrasound image sequence includes multiple sets of complex image sequences arranged in sequence. As shown in FIG. 4, the ultrasound imaging method further includes step S410 and step S420.

In step S410, coherent compounding is performed on the multiple sets of complex image sequences in the ultrasound image sequence to obtain relation image sequences.

In step S420, for the related area, the relation image sequences are processed separately to obtain a related-area image sequence capable of imaging the related area.

Further, in an embodiment of the present disclosure, the ultrasound imaging method further includes step S430.

In step S430, based on the to-be-imaged area, the target-area image sequence and the related-area image sequence are spliced to obtain a to-be-imaged-area complex image sequence.

Further, in an embodiment of the present disclosure, the ultrasound imaging method further includes step $440.

In step S440, imaging is performed on the to-be-imaged-area complex image sequence to obtain a to-be-imaged-area ultrasound image.

Specifically, the to-be-imaged area may include both the target area and the related area, the to-be-imaged-target volume of the target area is smaller than the to-be-imaged-target volume of the related area. For example, the to-be-imaged area may be a kidney area, and the target area may be a minute-vessel area in the kidney area, the related area may be a great-vessel area in the kidney area. In an embodiment, the conventional coherent compounding may be performed on the complex image sequences to obtain the relation image sequences. Further, for the related area, the relation image sequences are processed separately to obtain the related-area image sequence capable of imaging the related area. Further, based on a relationship between the target area and the related area in the to-be-imaged area, the obtained target-area image sequence and the related-area image sequence may be spliced to obtain a to-be-imaged-area complex image sequence. The imaging process, for example, a post-processing operation for a conventional image sequence, is performed on the to-be-imaged-area complex image sequence, to obtain an image of the to-be-imaged area, and the image of the to-be-imaged area may be an ultrasonic micro-blood flow image of the to-be-imaged area. Through the above-mentioned method of dividing the to-be-imaged area into the target area and the related area, for the related area, the relation image sequences of the related area with a relatively large to-be-imaged-target volume may be further processed based on the conventional coherent compounding processing, thereby reducing the amount of calculation and improving processing efficiency. In addition, based on the cyclic polling-based coherent compounding, and based on the processing performed on the target image sequences for the target area with a relatively small to-be-imaged-target volume, the imaging frame rate and the signal strength for the to-be-imaged target with a relatively small volume may be effectively improved, thus reducing noise. The image of the to-be-imaged area is obtained finally by splicing, and the images of the to-be-imaged targets of different volumes have relatively high resolutions, thereby effectively detecting a relatively small to-be-imaged target in the to-be-imaged area.

In some examples, the related area RI and the target area R2 in the to-be-imaged area may be divided by means of frame selection, where the target area R2 is a region of interest for which a clutter inhibition is required. Furthermore, the conventional coherent compounding may be performed as shown in FIG. 5(a). For example, for the single-angle unfocused wave images IQData(p,t) arranged in sequence in the ultrasound image sequence and divided into multiple sets of complex image sequences IQData(t), the conventional coherent compounding is performed on each set of complex image sequence IQData(t) to obtain the relation image sequences IQDataN(t). For example, the single-angle unfocused wave images IQData(p, t) in the complex image sequence IQData(t) may include images IQData(1,1), IQData(2,1), IQData(3,1) and IQData(4,1), where p=1,2,3 and 4, and t=1. For example, the conventional coherent compounding may be performed on the complex image sequence IQData(t) to obtain the relation image sequence IQDataN(t). Further, for the related area R1, the relation image sequence IQDataN(t) is further processed, such as filtered, to obtain the related-area image sequence I1(t). As shown in FIG. 5(b), the cyclic polling-based coherent compounding may be performed according to the method of step S310 to step S320. Multiple target image sequences IQDataH(t) may be determined by selecting from the ultrasound image sequence, and each target image sequence IQDataH(t) includes multiple single-angle unfocused wave images IQData(p,t) which are adjacent in sequence in the ultrasound image sequence, for example, the target image sequence IQDataH(t) may include images IQData(1,1), IQData(2,1), IQData(3,1) and IQData(4,1), and the target image sequence IQDataH(2) may include images IQData(2,1), IQData(3,1), IQData(4,1) and IQData(1,2). And so forth, the same single-angle unfocused wave image may be included in adjacent target image sequences. Further, the multiple target image sequences IQDataH(t) are processed separately for the target area R2, to obtain the target-area image sequence I2(t) capable of imaging the target area. Further, the obtained related-area image sequence I1(t) and the obtained target area image sequence I2(t) may be spliced to obtain the to-be-imaged-area complex image sequences I(t). The imaging process is performed on the to-be-imaged-area complex image sequences I(t). For example, the to-be-imaged-area complex image sequence I(t) is multiplied by a conjugate thereof, and then the product is summed along the time dimension, and a logarithmic compression is performed to obtain the final to-be-imaged-area ultrasound image, i.e. the ultrasound micro-blood flow image.

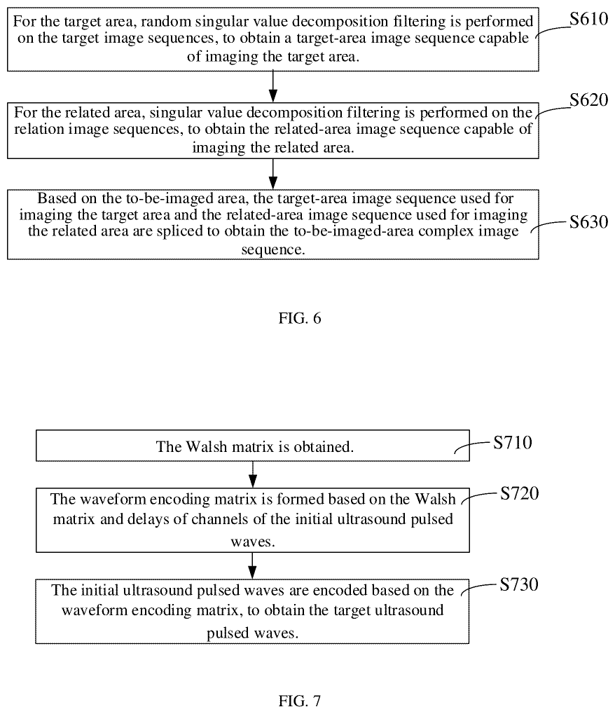

In an embodiment, the to-be-imaged area further includes the related area. As shown in FIG. 6, the ultrasound imaging method further includes step S610 to step S630.

In step S610, for the target area, random singular value decomposition filtering is performed on the target image sequences, to obtain a target-area image sequence capable of imaging the target area.

In step S620, for the related area, singular value decomposition filtering is performed on the relation image sequences, to obtain the related-area image sequence capable of imaging the related area.

Further, the ultrasound imaging method of the present disclosure further includes step S630. In step S630, based on the to-be-imaged area, the target-area image sequence used for imaging the target area and the related-area image sequence used for imaging the related area are spliced to obtain the to-be-imaged-area complex image sequence.

Specifically, the target image sequence may include multiple high-frame-rate compound unfocused wave complex images. The high-frame-rate compound unfocused wave complex images may include at least one of the high-frame-rate compound plane wave complex image and the high-frame-rate compound divergent wave complex image. The random singular value decomposition filtering may be performed on the multiple target image sequences. For example, a random singular value decomposition filter is used to filter multiple high-frame-rate compound unfocused wave complex images, to obtain a target-area image sequence for imaging the target area, for example, obtain a complex image sequence of tiny vessels. The conventional coherent compounding may be performed on each set of complex image sequence in the ultrasound image sequence to obtain the multiple relation image sequences. Each relation image sequence may include several compound unfocused wave complex images. The compound unfocused wave complex images may include at least one of the compound plane wave complex image and the compound divergent wave complex image. Further, for the related area, the singular value decomposition filtering may be performed on the relation image sequences. For example, a singular value decomposition filter is used to filter several compound unfocused wave complex images, to obtain the related-area image sequence used for imaging the related area. The related-area image sequence may be a complex image sequence of the vessels. According to the above method, the to-be-imaged area may be divided into the target area and the related area according to requirements of imaging. For example, the related-area image sequence for imaging the related area with a larger to-be-imaged-target volume is obtained based on the conventional coherent compounding, thereby reducing the amount of calculation and improving the processing efficiency. The target-area image sequence for imaging the target area with a smaller to-be-imaged-target volume is obtained based on the cyclic polling-based coherent compounding, thereby effectively improving the frame rate of imaging and the signal strength, and reducing noises. After the target image sequences and the relation image sequences are filtered respectively, the target-area image sequence obtained for the target area and the related-area image sequence obtained for the related area are spliced. The singular value decomposition filter takes advantages of different features of the tissues and the blood movement in terms of spatiotemporal coherence to provide higher-dimensional feature information, which can improve the sensitivity to the targets, for example, great vessels or minute vessels in the area. The random singular value decomposition filter may reduce the dimension of the target image sequence stably and fast, and the performance thereof does not depend on local features, thus further improving the efficiency of processing the tiny vessels and the sensitivity to the tiny vessels. The to-be-imaged-area complex image sequence obtained finally by splicing can improve the clarity of both the target-area image sequence and the related-area image sequence for imaging.

In some examples, as shown in FIG. 5(b), the target image sequences include 397 high-frame-rate compound unfocused wave complex images. For the target area R2 representing the tiny vessel area, a random singular value decomposition filter may be used to analyze the 397 high-frame-rate compound unfocused wave complex images IQDataH(t), to obtain the target-area image sequence for the target area, i.e., the complex image sequence 12(t) of the tiny vessels. As shown in FIG. 5(a), the relation image sequences include 100 compound unfocused wave complex images. For the related area RI representing the great vessel area, a singular value decomposition filter can be used to analyze 100 compound unfocused wave complex images IQDataN(t), to obtain the related-area image sequence used for imaging the related area, i.e., the complex image sequences I1(t) of the great vessels. I1(t) and I2(t) are placed at the positions corresponding to the related area R1 and the target area R2 respectively, and are spliced to form the to-be-imaged-area complex image sequence, i.e., the final complex image sequence I(t) of the micro-blood flow.

In an embodiment, as shown in FIG. 7, the step S110 of encoding the initial ultrasound pulsed waves to obtain the target ultrasound pulsed waves, includes steps S710 to S730. In step S710, the Walsh matrix is obtained.

In step S720, the waveform encoding matrix is formed based on the Walsh matrix and delays of channels of the initial ultrasound pulsed waves.

In step S730, the initial ultrasound pulsed waves are encoded based on the waveform encoding matrix, to obtain the target ultrasound pulsed waves.

Specifically, the Walsh matrix is obtained, and the waveform encoding matrix is formed based on the Walsh matrix and delays of channels of the initial ultrasound pulsed waves. For example, the waveform encoding matrix is formed, based on the convolution of the delays of the channels corresponding to the inclination angles of the unfocused waves and the Walsh matrix. The delays of channels of the initial ultrasound pulsed waves may be determined based on the transmission angles of the initial ultrasound pulsed waves, namely the unfocused waves. The Walsh matrix may be a square matrix with a dimension M, where M is a natural number. The Walsh matrix consists of −1 and 1, and any two rows or any two columns thereof are orthogonal, i.e., have the scalar product equal to zero. The order M of the Walsh matrix may be determined based on the number of transmission angles. The initial ultrasound pulsed waves may be determined based on the transmission parameters of the initial ultrasound pulsed waves. For example, the initial ultrasound pulsed waves may be determined based on the transmission parameters of the multi-angle coherent compounding unfocused waves. The initial ultrasound pulsed waves may be multi-angle coherent compounding unfocused waves. The transmission parameters may include the center frequency of the transmission pulse, the number of cycles of the transmission pulse, the initial phase of the transmission pulse, the number of transmission angles, and the maximum transmission angle. Based on the number of transmission angles and the maximum transmission angle, the transmission delay of the transmission pulse can be determined. Before forming the waveform encoding matrix based on the Walsh matrix and the delays of the channels of the initial ultrasound pulsed waves, the method further includes determining the delays of the channels of the initial ultrasound pulsed waves based on the number of the transmission angles and the maximum transmission angle of the initial ultrasound pulsed waves. The transmit delay of the transmit pulse may also be determined based on the number of transmission angles, the maximum transmission angle, and parameters of the transducer. Further, the initial ultrasound pulsed waves may be determined based on the center frequency of the transmit pulse, the number of cycles of the transmission pulse, the initial phase of the transmit pulse, and the number of cycles of the transmission pulse.

Further, the target ultrasound pulsed waves may be obtained by encoding the initial ultrasound pulsed waves based on the waveform encoding matrix. The waveform encoding matrix may be generated based on the Walsh matrix W(p, q) as follows:

$$W ( p , q ) = {\prod }_{k = 0}^{M - 1} {[ R ( k + 1 , q ) ]}^{{g ( p )}_{k}}$$

where R(k+1,q) denotes any Rademaker function, g(p) denotes a Gray code of p, g(p) k denotes a k-th bit of the Gray code g(p), p denotes the index number of the number of times the initial ultrasound pulsed waves transmit, p=1,2,3 . . . , 2^{na}, M denotes the order of the Walsh matrix, and M=2^{na}, and q denotes a continuous time variable. The waveform encoding matrix is obtained in the above way, may be used for encoding the initial ultrasound pulsed waves. For example, the initial ultrasound pulsed waves are encoded based on the principle of CDM in the communication field to determine the target ultrasound pulsed waves. The total length of the obtained target ultrasound pulsed waves is M times of the length of the initial ultrasound pulsed waves, where M represents the order of the waveform encoding matrix. The target ultrasound pulsed waves obtained based on the above manner are transmitted to the to-be-imaged area, and the number of cycles of the transmission pulse can be increased, thereby greatly improving the penetration ability of the unfocused waves in deeper parts of the tissues, and preventing the signals of deep micro-blood flow from being submerged in noise. Multiple sets of the ultrasound echo data corresponding to the target ultrasound pulsed waves are obtained, thus the intensity of the ultrasound echo data which reflect the blood flow echo signals is increased at the front end, and the noise level is inhibited.

In some examples, the initial ultrasound pulsed waves can be TWB(i, p), where i denotes an index number of array elements of the transducer, i=1,2,3 . . . , N, where N denotes the number of the array elements of the ultrasonic transducer, j denotes an index number of the initial transmission angle, j=1,2,3 . . . , 2^{na}, 2^{na} denotes the number of transmission angles, na may be any one of 1, 2, 3 or 4, and N_{TW} denotes the length of the initial ultrasound pulsed waves TWA(i, j) TWB(i, p).

The target ultrasound pulsed waves may be determined based on the waveform encoding matrix and the initial ultrasound pulsed waves. For example, based on Walsh matrix W(p, q) used to determine the waveform encoding matrix, and the initial ultrasound pulsed waves TWA(i, j) ultrasound pulsed waves TWB(i, p) may be determined, where the total length of the ultrasound pulsed waves is 2^{na}×N_{TW}, and the ultrasound pulsed waves are divided into 2^{na} segments in time sequence, and each segment consists of a product of a corresponding initial ultrasound pulsed wave TWA(i, j) and the Walsh matrix W(p, q), that is:

$$TWB ( i , p ) [ ( 1 + ( j - 1 ) \times {N}_{TW} ) to j \times {N}_{TW} ] = TWA ( i , j ) \times W ( p , q )$$

where TWB(i, p) represents the target ultrasound pulsed waves, i represents the index number of an array element of the transducer, j represents the index number of the initial transmission angle, p represents the index number of the number of times of transmissions, N_{TW} represents the length of the initial ultrasound pulsed waves TWA(i, j), TWA(i, j) represents the initial ultrasound pulsed waves, and W(p, q) represents the Walsh matrix.

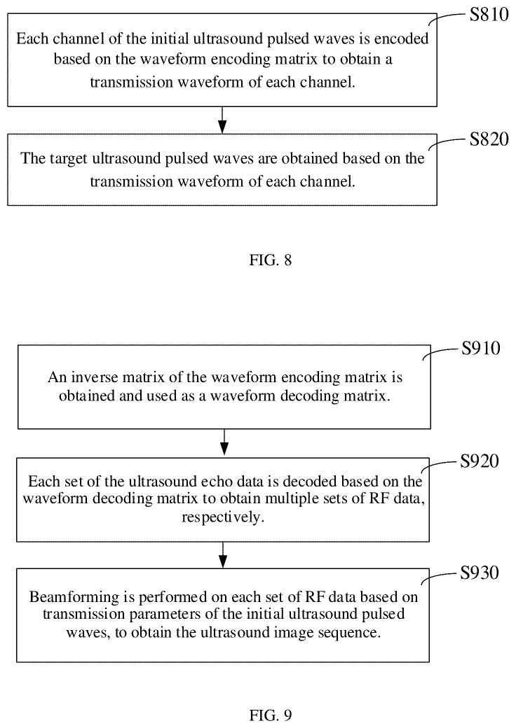

In an embodiment, as shown in FIG. 8, step S730 of encoding the initial ultrasound pulsed waves based on the waveform encoding matrix, to obtain the target ultrasound pulsed waves, includes steps S810 and S820.

In step S810, each channel of the initial ultrasound pulsed waves is encoded based on the waveform encoding matrix to obtain a transmission waveform of each channel.

In step S820, the target ultrasound pulsed waves are obtained based on the transmission waveform of each channel.

Specifically, according to the initial ultrasound pulsed waves and the waveform encoding matrix, the transmission waveforms of multiple channels may be obtained and used as the target ultrasound pulsed waves, and the length of the transmission waveform of each channel is the same as the length of the initial ultrasound pulsed waves. The transmission waveforms of multiple channels are composed of the product of the initial ultrasound pulsed waves and the waveform encoding matrix. By using the transmission waveforms of the multiple channels as the target ultrasound pulsed waves transmitted to the to-be-imaged area, the number of cycles of the transmission pulse can be increased, thereby greatly improving the penetration ability of unfocused waves in relatively deep parts of the tissues, and avoiding the attenuation of the echo signals of the blood flow during transmission in the tissues, and prevents the signals of the deep minute blood flow from being submerged in noises.

In some examples, the order M of the Walsh matrix may be 2^{na}, the order M of the Walsh matrix may be equal to the number of the transmission angles. For example, the Walsh matrix can be a square matrix of order 4, and the waveform encoding matrix may be determined based on the Walsh matrix of order 4.

In an embodiment, as shown in FIG. 9, step S120 of decoding the multiple sets of the ultrasound echo data to obtain the ultrasound image sequence includes steps S910 to S930.

In step S910, an inverse matrix of the waveform encoding matrix is obtained and used as a waveform decoding matrix.

In step S920, each set of the ultrasound echo data is decoded based on the waveform decoding matrix to obtain multiple sets of RF data, respectively.

In step S930, beamforming is performed on each set of RF data based on transmission parameters of the initial ultrasound pulsed waves, to obtain the ultrasound image sequence.

Specifically, the inverse matrix of the waveform encoding matrix may be obtained and used as the waveform decoding matrix. The waveform decoding matrix may be the inverse matrix of the waveform encoding matrix, or may be a matrix obtained based on the inverse matrix of the waveform encoding matrix. Based on the waveform decoding matrix, each set of ultrasound echo data may be decoded. For example, the ultrasound echo data RFA(i, j) may be decoded according to the inverse matrix of the waveform encoding matrix, to obtain decoded multiple sets of RF data RFB(i, p). For example, multiple sets of RF data RFB(i, p) may be obtained based on the inverse matrix W′ of the Walsh matrix as follows:

$$RFB ( i , p ) = {\sum }_{j = 1}^{M} RFA ( i , j ) \times {W}^{′} ( p , j )$$

where RFB(i, p) represents the RF data, RFA(i, j) represents the ultrasound echo data, and W′(p,j) represents the inverse matrix of the Walsh matrix.

Further, the multiple sets of RF data may be beamformed separately to obtain the ultrasound image sequence. For example, the multiple sets of RF data are separately beamformed based on the transmission parameters to obtain the ultrasound image sequence. Corresponding to the encoded target ultrasound pulsed waves, the ultrasound echo data may be decoded by the above method, which can enhance the intensity of the echo signal at each individual angle after echo decoding and ensure that the axial resolution of the image is not affected by the increase in the number of cycles of the transmission pulse, thus the intensity of the ultrasound echo data which reflects the echo signals of the blood flow is increased at the front end, and the noise level is inhibited.

In some examples, the following formula may be used to obtain the decoded RF data RFB(1,2) corresponding to the second transmission of the first array element:

$$RFB ( 1 , 2 ) = {\sum }_{j = 1}^{4} RFA ( 1 , j ) \times {W}^{′} ( 2 , j ) = RFA ( 1 , 1 ) + RFA ( 1 , 2 ) - RFA ( 1 , 3 ) - RFA ( 1 , 4 ) .$$

Further, the order of the single-angle unfocused wave images in a complex image sequence can be determined based on the transmission angle sequence. The transmission parameters may include the number of transmission angles and the maximum transmission angle. The transmission angle sequence may be determined based on the number of transmission angles and the maximum transmission angle. The number of the single-angle unfocused wave images in each target image sequence may be the same as the number of the single-angle unfocused wave images in the complex image sequence.

Specifically, the number of transmission angles may be 2^{na}, and na may be one of 1,2, 3, or 4. The maximum transmission angle may be any value from 3 degrees to 24 degrees. A transmission angle sequence may be determined based on the number of transmission angles and the maximum transmission angle, and the transmission angle sequence may include a specific number of transmission angles arranged in sequence. Further, the single-angle unfocused wave images may be sorted based on the transmission angle sequence, to obtain a corresponding complex image sequence. The complex image sequence may include the single-angle unfocused wave images of the number of transmission angles. As shown in FIG. 5(b), the number of the single-angle unfocused wave images in the target image sequence and the number of the single-angle unfocused wave images in the complex image sequence each may be the same as the number of transmission angles, for example, 4.

In some examples, the transmission parameters of the multi-angle coherent compounding unfocused wave may further include the center frequency of the transmission pulse, the number of cycles of the transmission pulse, and the initial phase of the transmission pulse. The center frequency of the transmission pulse may be any value from 1 MHz to 5 MHz. The number of cycles of the transmission pulse can be set to be any value from 1 to 5. The initial phase of the transmit pulse may be any value from 0 to 180 degrees. For example, the number of transmission angles is 4, the maximum transmission angle is 6 degrees, the center frequency of the transmission pulse is 3.5 MHz, the number of cycles of the transmission pulse is 2, the initial phase of the transmission pulse is zero, and the number of array elements of the ultrasonic transducer is 128, and the length N_{TW} of the initial ultrasound pulsed waves TWA(i, j) is 800 points (each point is 0.004 us), where i=1, 2, 3, . . . , 128, and j=1, 2, 3, 4.Correspondingly, the waveform encoding matrix may be determined based on the Walsh matrix W of four order as follows:

$$W = [ \begin{matrix}1 & 1 & 1 & 1 \\ 1 & 1 & - 1 & - 1 \\ 1 & - 1 & 1 & - 1 \\ 1 & - 1 & - 1 & 1\end{matrix} ]$$

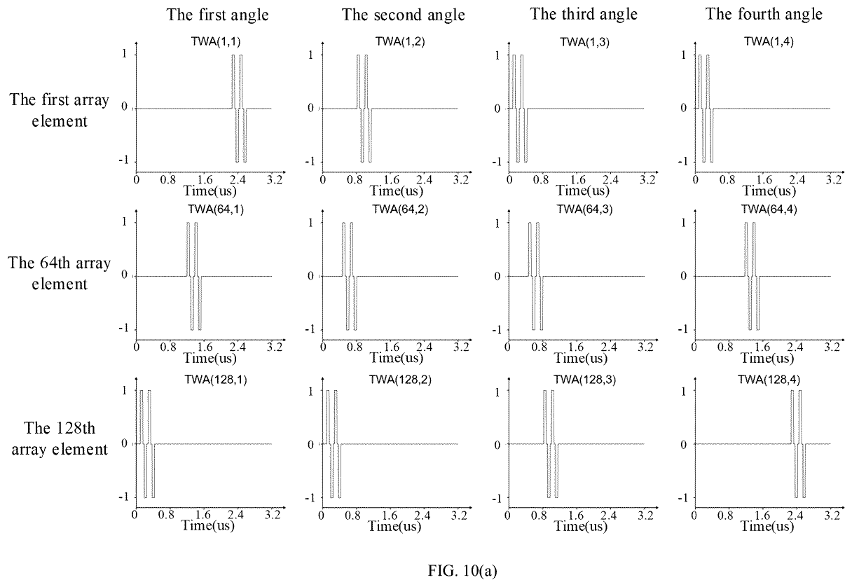

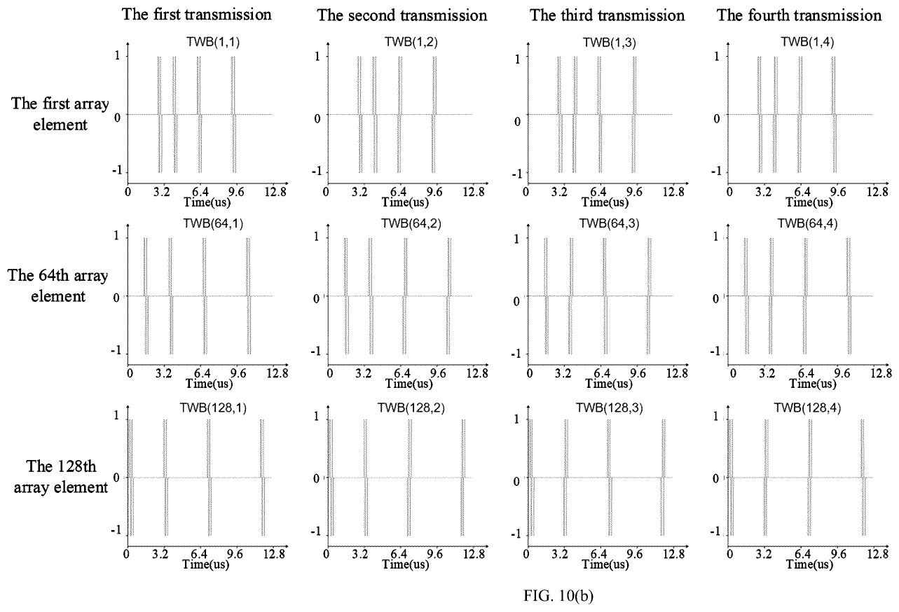

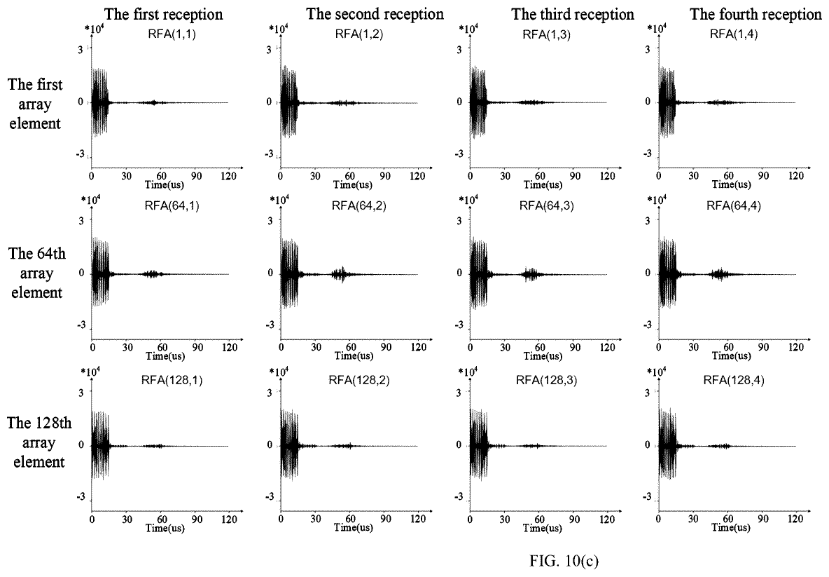

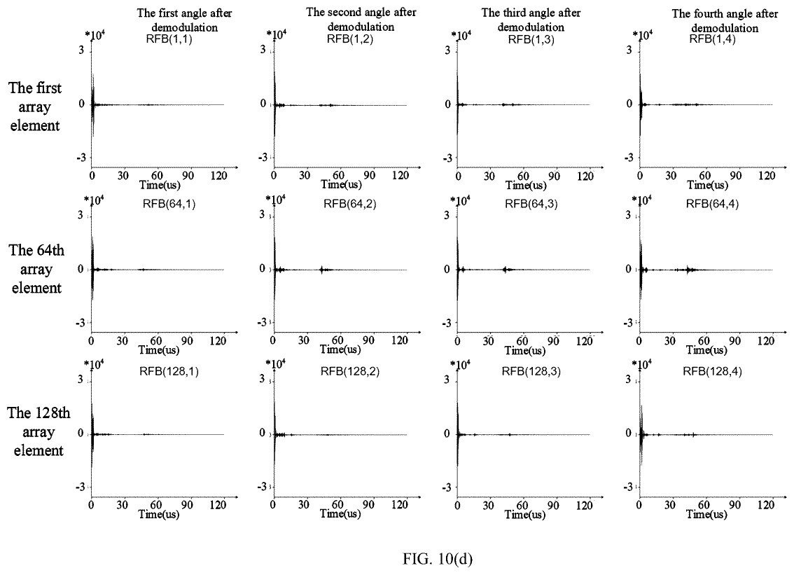

Based on the above transmission parameters, the initial ultrasound pulsed waves TWA(i, j) of an array element 1, an array element 64 and an array element 128 are as shown in FIG. 10(a). The target ultrasound pulsed waves TWB(i, p) of the array element 1, the array element 64 and the array element 128 are as shown in FIG. 10(b) TWB(i, p). FIG. 10(c) shows the ultrasound echo data RFA(i, j) of the array element 1, the array element 64 and the array element 128. FIG. 10(d) shows the decoded RF data RFB(i, p) of the array element 1, the array element 64 and the array element 128. For consecutive 100 sets of multi-angle coherent compounding unfocused waves, after beamforming based on the above-mentioned transmission parameters, 400 single-angle unfocused wave images IQData(p, t) may be obtained. Furthermore, through the cyclic polling-based coherent compounding as shown in FIG. 5(b), the high-frame-rate compound unfocused wave complex images IQDataH(t) with a frame rate increased by four times may be obtained, where t=1, 2, 3, . . . , 397.

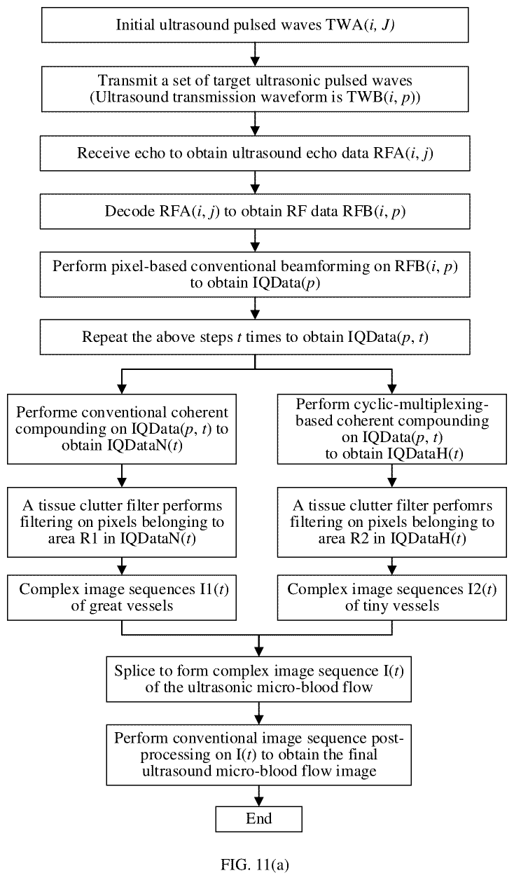

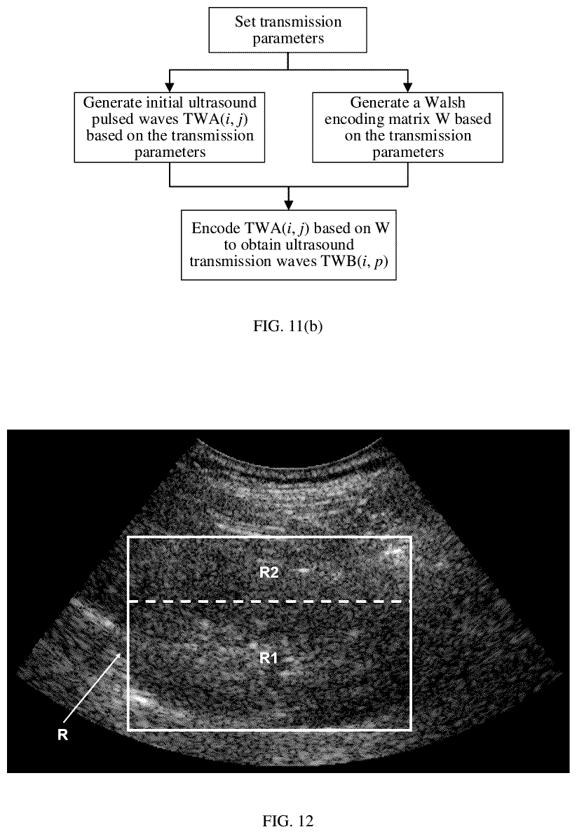

In some examples, as shown in FIG. 11(a), the ultrasound pulsed waves TWB(i, p) TWB(i, p) may be determined firstly. Specifically, the total length of the target ultrasound pulsed waves TWB(i, p) may be 3200 points. The target ultrasound pulsed waves are divided into four segments in time sequence, each segment consists of the product of the initial ultrasound pulsed waves TWA(i, j) and the Walsh matrix W as follows:

$$TWB ( i , p ) [ ( 1 + ( j - 1 ) \times 800 ) to j \times 800 ] = TWA ( i , j ) \times W ( p , j ) ,$$

where i denotes an index number of an array element of the transducer, j denotes the index number of the initial transmission angle, p denotes the index number of the number of times of transmissions, i=1, 2, 3, . . . , 128, j=1, 2, 3, 4, and p=1, 2, 3, 4.

As shown in FIG. 11(b), the initial ultrasound pulsed waves TWA(i, j) and the waveform encoding matrix namely the Walsh encoding matrix W may be generated by setting the transmission parameters. Based on the above formula, the initial ultrasound pulsed waves TWA(i, j) are encoded according to the waveform encoding matrix to obtain the target ultrasound pulsed waves TWB(i, p) to be transmitted finally.

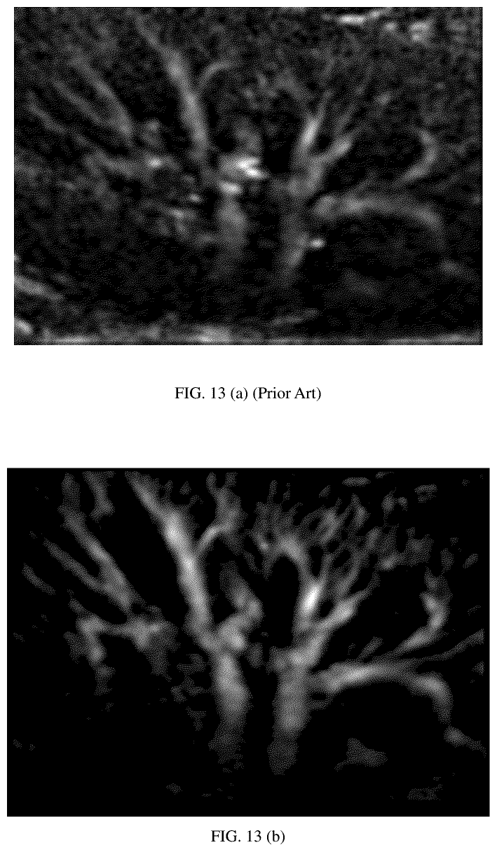

Further, based on the target ultrasound pulsed waves TWB(i, p), a set of ultrasonic pulses are transmitted and echo signals corresponding to the ultrasonic pulses are received, to obtain the ultrasound echo data RFA(i, j). The ultrasound echo data RFA(i, j) are decoded to obtain the RF data RFB(i, p). Pixel-based conventional beamforming is performed on the RF data RFB(i, p) to obtain the corresponding complex image sequence IQData(p). The above steps are repeated for t times, i.e., t sets of ultrasonic pulses are transmitted, to obtain the ultrasound image sequence IQData(p,t). The conventional coherent compounding is performed on the single-angle unfocused wave image IQData(p,t) in the ultrasound image sequence, to obtain the relation image sequence IQDataN(t). As shown in FIG. 12, the to-be-imaged area (the region of interest R), for which a clutter inhibition is required, may be manually selected through a frame, and the to-be-imaged area is divided into the related area R1 (namely the great vessel area R1) and the target area R2 (namely the tiny vessel area R2). The tissue clutter filter is used to perform filtering on the pixels belonging to the related area R1 in the relation image sequence IQDataN(t), to obtain the related-area image sequence, i.e., the complex image sequence I1(t) of the great vessels. The cyclic polling-based coherent compounding is performed on the single-angle unfocused wave image IQData(p, t) in the ultrasound image sequence, as shown in FIG. 5(b), to obtain the target image sequence IQDataH(t). The tissue clutter filter is used to perform filtering on the pixels belonging to the target area R2 in the target image sequence IQDataH(t), to obtain the target-area image sequence, i.e., the complex image sequence I2(t) of the tiny vessels. The complex image sequence I1(t) of the great vessels and the complex image sequence I2(t) of the tiny vessels are spliced, to obtain the to-be-imaged-area complex image sequences I(t), namely an ultrasound micro-blood-flow complex image sequence I(t). After the conventional image sequence post-processing is performed on the ultrasound micro-blood-flow complex image sequence I(t), the final ultrasonic micro-blood flow image is obtained and outputted for display. For example, the ultrasound micro-blood-flow complex image sequence I(t) is multiplied by its own conjugate, and then is summed along the time dimension, and the logarithmic compression is performed to obtain the final to-be-imaged-area ultrasound image. FIG. 13(a) shows an ultrasonic blood flow image of the kidney by a conventional method in the prior art. FIG. 13(b) shows an ultrasonic micro-blood flow image of the kidney according to an embodiment of the present disclosure. Compared with the existing technical solutions, the method of the present disclosure can reduce noises of the ultrasonic micro-blood flow image and improve the frame rate. The above-mentioned ultrasound imaging method improves the steps of transmitting, receiving, and beamforming ultrasonic unfocused waves based on the principle of CDM, and enhances the penetration ability of the unfocused waves in the deep tissues and increases the detectable range of the minimum blood flow without sacrificing the image axial resolution and the frame rate of imaging, thereby effectively retaining the echo signals of the micro-blood flow in the deep issues. After the tissue clutter filter performs filtering on the target image sequence and on the relation image sequence, the obtained target-area image sequence and the related-area image sequence are spliced to image, thereby clearly imaging the micro-blood flow in the deep organs and tissues.

It should be understood that although the steps in the flowcharts involved in the above embodiments are shown in sequence as indicated by the arrows, these steps are not necessarily executed in the order indicated by the arrows. Unless explicitly stated in this disclosure, there is no strict order restriction on the execution of these steps, and these steps can be executed in other orders. Moreover, at least some of the steps in the flowcharts involved in the above embodiments may include multiple steps or stages. These steps or stages are not necessarily executed at the same time, but may be executed at different times. The execution order of these steps or stages is not necessarily sequential, but may be performed in turn or alternately with other steps or at least part of the steps or stages in other steps.

Based on the same inventive concept, embodiments of the present disclosure also provide an ultrasound imaging device for implementing the above-mentioned ultrasound imaging method. The solutions to the problem provided by this device are similar to the solutions described in the above method. Therefore, for the specific limitations of one or more embodiments of the ultrasound imaging device provided hereinafter, please refer to the limitations of the ultrasound imaging method above, which will not described repeatedly herein.

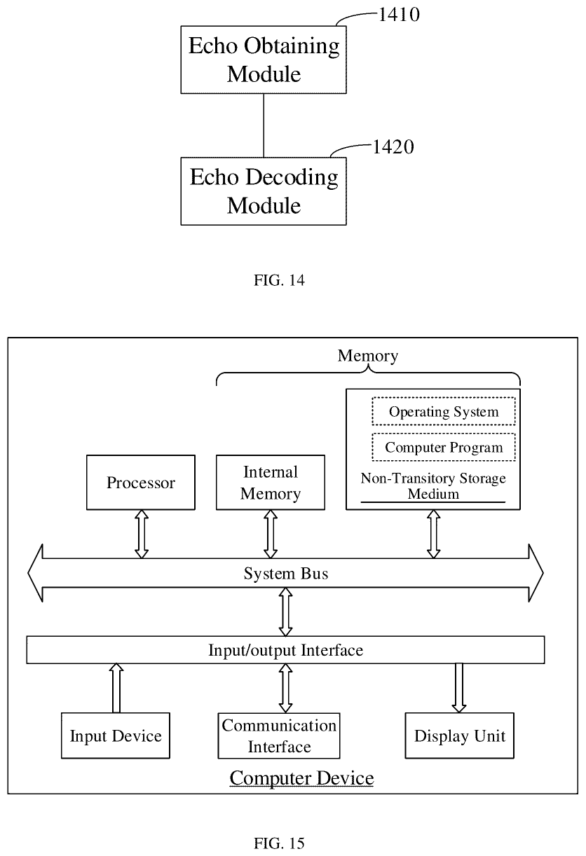

In an embodiment, as shown in FIG. 14, an ultrasound imaging device is provided. The device includes an echo obtaining module 1410 and an echo decoding module 1420.

The echo obtaining module 1410 is configured to encode initial ultrasound pulsed waves to obtain target ultrasound pulsed waves, and obtain multiple sets of ultrasound echo data of a to-be-imaged area based on the target ultrasound pulsed waves. The number of cycles of each of the target ultrasound pulsed waves is greater than the number of cycles of each the initial ultrasound pulsed waves.

The echo decoding module 1420 is configured to decode multiple sets of the ultrasound echo data to obtain an ultrasound image sequence.

In an embodiment, the to-be-imaged area includes a target area. The ultrasound imaging device further includes a coherent compounding module.

The coherent compounding module is configured to perform cyclic polling-based coherent compounding on the ultrasound image sequence, to obtain a plurality of target image sequences. Each target image sequence includes multiple single-angle unfocused wave images that are sequentially adjacent in the ultrasound image sequence. The coherent compounding module is configured to process, for the target area, the plurality of target image sequences separately, to obtain a target-area image sequence capable of imaging the target area.

In an embodiment, the coherent compounding module is also configured to select a current target image sequence from single-angle unfocused wave images in the ultrasound image sequence, and configured to select a next target image sequence from single-angle unfocused wave images in the ultrasound image sequence based on the current target image sequence, until the plurality of target image sequences are obtained. The next target image sequence contains at least one of the single-angle unfocused wave images in the current target image sequence.

In an embodiment, the to-be-imaged area further includes a related area. The ultrasound imaging device further includes a target-image-sequence processing module, a relation-image-sequence processing module, and a splicing module.

The target-image-sequence processing module is configured to perform random singular value decomposition filtering on the target image sequences, to obtain a target-area image sequence capable of imaging the target area.

The relation-image-sequence processing module is configured to singular value decomposition filtering on the relation image sequences, to obtain the related-area image sequence capable of imaging the related area.

The splicing module is configured to splice the target-area image sequence and the related-area image sequence to obtain a to-be-imaged-area complex image sequence.

In an embodiment, the ultrasound imaging device further includes a Walsh matrix obtaining module, a waveform encoding matrix obtaining module, and a target ultrasonic pulse obtaining module.

The Walsh matrix obtaining module is configured to obtain a Walsh matrix.

The waveform encoding matrix obtaining module is configured to form a waveform encoding matrix based on the Walsh matrix and delays of channel of the initial ultrasound pulsed waves.

The target ultrasonic pulse obtaining module is configured to encode the initial ultrasound pulsed waves, based on the waveform encoding matrix, to obtain the target ultrasound pulsed waves.

In an embodiment, the echo obtaining module 1410 is also configured to encode each channel of the initial ultrasound pulsed waves based on the waveform encoding matrix, to obtain a transmission waveform of each channel, and to obtain the target ultrasound pulsed waves based on the transmission waveform of each channel.

In an embodiment, the echo decoding module 1420 is also configured to obtain an inverse matrix of the waveform encoding matrix used as a waveform decoding matrix; decode each set of the ultrasound echo data based on the waveform decoding matrix to obtain multiple sets of RF data respectively; and perform beamforming on each set of RF data, based on transmission parameters of the initial ultrasound pulsed waves, to obtain the ultrasound image sequence.

Each module in the above-mentioned ultrasound imaging device may be implemented in whole or in part by software, hardware, and a combination thereof. Each of the above modules may be embedded in or independent of the processor of the computer device in the form of hardware, or may be stored in the memory of the computer device in the form of software, so that the processor may call and execute the operations corresponding to the above modules.

In an embodiment, a computer device is proposed, and includes a memory and a processor. The memory stores a computer program. The processor, when executing the computer program, performs the steps of the methods above.

In an embodiment, a computer device is provided. The computer device may be a terminal, and its internal structure diagram may be as shown in FIG. 15. The computer device includes a processor, memory, input/output interface, communication interface, display unit and input device. Among them, the processor, memory and input/output interface are connected through the system bus, and the communication interface, display unit and input device are connected to the system bus through the input/output interface. The processor of the computer device is used to provide computing and control capabilities. The memory of the computer device includes non-volatile storage media and internal memory. The non-volatile storage medium stores operating systems and computer programs. This internal memory provides an environment for the execution of operating systems and computer programs in the non-volatile storage media. The input/output interface of the computer device is used to exchange information between the processor and external devices. The communication interface of the computer device is used for wired or wireless communication with external terminals. The wireless mode can be implemented through WIFI, mobile cellular network, NFC (Near Field Communication) or other technologies. The computer program, when executed by the processor, implements an ultrasound imaging method. The display unit of the computer device is used to form a visually visible picture, and may be a display screen, a projection device or a virtual reality imaging device. The display screen can be a liquid crystal display or an electronic ink display. The input device of the computer device can be a touch layer covering the display screen, or it can be a button, trackball or touch pad provided on the computer device casing, or it can be an external keyboard, a trackpad or a mouse, etc.

Those skilled in the art can understand that the structure shown in FIG. 15 is only a block view showing a partial structure related to the solutions of the present disclosure, and does not constitute a limitation on the computer device to which the solutions of the present disclosure are applied. Specific computer device may include more or fewer parts than those are shown, or may combine some parts, or may have a different arrangement of parts.

In an embodiment, an ultrasound imaging system is proposed. The system includes a waveform generator and an ultrasonic transducer which are capable of generating and transmitting the target ultrasound pulsed waves. The waveform generator is configured to generate electrical signals, and the ultrasonic transducer is configured to realize a conversion from electrical signals to sound waves. The waveform generator may be a discrete waveform generator with 3 levels, 5 levels, or a higher number of levels, or may be a waveform generator for generating a continuously changing linear waveform. The system further includes a computer device connected to the waveform generator and the ultrasonic transducer, and the steps of the above method are implemented when the computer device executes the computer program.

An embodiment proposes a non-transitory computer-readable storage medium, on which a computer program is stored, and when the computer program is executed by a processor, the steps of the above method are implemented.

An embodiment proposes a computer program product, which includes an executable program. The program, when executed by a processor, implements the steps of the above method.

Those of ordinary skill in the art can understand that all or part of the processes in the methods of the above embodiments can be completed by instructing relevant hardware through a computer program. The computer program can be stored in a non-volatile computer-readable storage medium. When executed, the computer program may include the processes of the above method embodiments. Any reference to memory, database or other media used in the embodiments provided in the present disclosure may include at least one of non-volatile and volatile memory. The non-volatile memory may include read-only memory (ROM), magnetic tape, floppy disk, flash memory, optical memory, high-density embedded non-volatile memory, resistive memory (ReRAM), Magneto resistive Random Access Memory (MRAM), Ferroelectric Random Access Memory (FRAM), Phase Change Memory (PCM), graphene memory, etc. Volatile memory may include Random Access Memory (RAM) or external cache memory, etc. As an illustration and not a limitation, RAM can be in various forms, such as Static Random Access Memory (SRAM) or Dynamic Random Access Memory (DRAM). The databases involved in the various embodiments of the present disclosure may include at least one of a relational database and a non-relational database. The non-relational database may include, but is not limited to, blockchain-based distributed database, etc. The processors involved in the various embodiments of the present disclosure may be, but are not limited to, a general-purpose processor, a central processing unit, a graphics processor, a digital signal processor, a programmable logic device, a quantum computing-based data processing logic device, etc.

The technical features of the embodiments above may be combined arbitrarily. To make the description concise, not all possible combinations of the technical features in the above embodiments are described. However, as long as there are no contradictions in the combinations of these technical features, all of the combinations should be considered to be within the scope of the specification.

The embodiments above only represent several implementation modes of the present application, and the description thereof is relatively specific and detailed, but it should not be construed as limiting the scope of the patent. It should be noted that for those skilled in the art, various modifications and improvements may be made without departing from the concept of the present application, and all these modifications and improvements belong to the protection scope of the present application. Therefore, the scope of protection of the patent application should be subject to the appended claims.

What is claimed is:

1. An ultrasound imaging method, comprising:
encoding initial ultrasound pulsed waves to obtain target ultrasound pulsed waves, and obtaining multiple sets of ultrasound echo data of a to-be-imaged area based on the target ultrasound pulsed waves, the number of cycles of each of the target ultrasound pulsed waves being greater than the number of cycles of each of the initial ultrasound pulsed waves; and
decoding the multiple sets of the ultrasound echo data to obtain an ultrasound image sequence.

2. The method according to claim 1, wherein:
the to-be-imaged area comprises a target area; and
the method further comprises:
performing cyclic polling-based coherent compounding on the ultrasound image sequence to obtain a plurality of target image sequences, each of the plurality of target image sequences comprising multiple single-angle unfocused wave images that are sequentially adjacent in the ultrasound image sequence; and
processing the plurality of target image sequences separately for the target area to obtain a target-area image sequence capable of imaging the target area.

3. The method according to claim 2, wherein, performing the cyclic polling-based coherent compounding on the ultrasound image sequence to obtain the plurality of target image sequences comprises:
determining a current target image sequence by selecting from single-angle unfocused wave images in the ultrasound image sequence; and
determining a next target image sequence by selecting from the single-angle unfocused wave images in the ultrasound image sequence based on the current target image sequence, until the plurality of target image sequences are obtained, wherein the next target image sequence comprises at least one of single-angle unfocused wave images in the current target image sequence.

4. The method according to claim 2, wherein:
the to-be-imaged area further comprises a related area; and
the method further comprises:
performing coherent compounding on multiple sets of complex image sequences in the ultrasound image sequence to obtain relation image sequences, each set of complex image sequences comprising multiple single-angle unfocused wave images; and
processing the relation image sequences separately for the related area to obtain a related-area image sequence capable of imaging the related area.

5. The method according to claim 4, further comprising: splicing the target-area image sequence and the related-area image sequence to obtain a to-be-imaged-area complex image sequence.

6. The method according to claim 5, further comprising: performing imaging on the to-be-imaged-area complex image sequence to obtain a to-be-imaged-area ultrasound image.

7. The method according to claim 6, wherein, performing imaging on the to-be-imaged-area complex image sequence to obtain the to-be-imaged-area ultrasound image comprises:
multiplying the to-be-imaged-area complex image sequence by a conjugate thereof; and
summing a product of the to-be-imaged-area complex image sequence and the conjugate thereof along the time dimension, and performing a logarithmic compression to obtain the to-be-imaged-area ultrasound image.

8. The method according to claim 5, wherein:
processing the plurality of target image sequences separately for the target area comprises: performing random singular value decomposition filtering on the plurality of target image sequences separately for the target area; or
processing the relation image sequences separately for the related area, comprises: performing singular value decomposition filtering on the relation image sequences separately for the related area.

9. The method according to claim 1, wherein encoding the initial ultrasound pulsed waves to obtain the target ultrasound pulsed waves, comprising:
obtaining a Walsh matrix;
forming a waveform encoding matrix based on the Walsh matrix and delays of channels of the initial ultrasound pulsed waves; and
encoding the initial ultrasound pulsed waves based on the waveform encoding matrix to obtain the target ultrasound pulsed waves.

10. The method according to claim 9, wherein before forming the waveform encoding matrix based on the Walsh matrix and the delays of channels of the initial ultrasound pulsed waves, the method further comprises:
determining the delays of channels of the initial ultrasound pulsed waves based on the number of transmission angles and a maximum transmission angle of the initial ultrasound pulsed waves.

11. The method according to claim 9, wherein the Walsh matrix is as follows:
$$W ( p , q ) = {\prod }_{k = 0}^{M - 1} {[ R ( k + 1 , q ) ]}^{{g ( p )}_{k}}$$
wherein R(k+1,q) denotes a Rademaker function, g(p) denotes a Gray code of p, g(p)ₖ denotes a k-th bit of the Gray code g(p), p denotes an index number of the number of times the initial ultrasound pulsed waves transmit, p=1, 2 to 2^{na}, na denotes any one of 1, 2, 3 or 4, M denotes an order of the Walsh matrix, M=2^{na}, and q denotes a continuous time variable.

12. The method according to claim 11, wherein the target ultrasound pulsed waves are as follows:
$$TWB ( i , p ) [ ( 1 + ( j - 1 ) \times {N}_{TW} ) to j \times {N}_{TW} ] = TWA ( i , j ) \times W ( p , q ) ,$$
where TWB(i, p) denotes the target ultrasound pulsed waves, i denotes an index number of an array element of a transducer, j denotes an index number of a transmission angle, TWA(i, j) denotes the initial ultrasound pulsed waves, and N_{TW} denotes a length of the initial ultrasound pulsed wavesTWA(i, j).

13. The method of claim 12, wherein the order M of the Walsh matrix is equal to the number of transmission angles of the initial ultrasound pulsed waves.

14. The method according to claim 9, wherein encoding the initial ultrasound pulsed waves based on the waveform encoding matrix to obtain the target ultrasound pulsed waves comprises:
encoding each channel of the initial ultrasound pulsed waves based on the waveform encoding matrix to obtain a transmission waveform of each channel; and
obtaining the target ultrasound pulsed waves based on the transmission waveform of each channel.

15. The method according to claim 9, wherein decoding the multiple sets of the ultrasound echo data to obtain the ultrasound image sequence comprises:
obtaining an inverse matrix of the waveform encoding matrix to serve as a waveform decoding matrix;
decoding each set of the multiple sets of ultrasound echo data based on the waveform decoding matrix to obtain multiple sets of radio frequency (RF) data, respectively; and
performing beamforming on each set of the multiple sets of RF data based on transmission parameters of the initial ultrasound pulsed waves to obtain the ultrasound image sequence.

16. The method according to claim 15, wherein:
the waveform decoding matrix is the inverse matrix of the Walsh matrix; and
the multiple sets of RF data are as follows:
$$RFB ( i , p ) = {\sum }_{j = 1}^{M} RFA ( i , j ) \times {W}^{′} ( p , j )$$
wherein RFB(i, p) denotes the RF data, RFA(i, j) denotes the ultrasound echo data, and W′(p, j) denotes the inverse matrix of the Walsh matrix.

17. The method according to claim 10, wherein the number of the transmission angles of the initial ultrasound pulsed waves is 2^{na}, wherein na is any one of 1, 2, 3 or 4; or the maximum transmission angle is any value from 3 degrees to 24 degrees.

18. A non-transitory computer-readable storage medium, comprising a computer program stored thereon, wherein the computer program, when executed by a processor, causes the processor to perform steps of the method of claim 1.

19. A computer device, comprising a memory and a processor, wherein the memory stores a computer program, and the processor, when executing the computer program, performs steps of the method of claim 1.

20. An ultrasound imaging system, comprising:
a waveform generator and an ultrasonic transducer, which are configured for generating and transmitting target ultrasound pulsed waves; and
a computer device connected to the waveform generator and the ultrasonic transducer, wherein the computer device, when executing a computer program, performs steps of the method of claim 1.
